(19) United States
(12) Patent Application Publication
(10) Pub. No.: US 2019/0247437 A1
(43) Pub. Date: Aug. 15, 2019

(54) ENGINEERED LYMPHOCYTE COMPOSITIONS, METHODS AND SYSTEMS

(71) Applicant: APDN (B.V.I.), Inc., Tortola (VG)

(72) Inventors: Stephen Hughes, Port Jefferson Station, NY (US); James A. Hayward, Stony Brook, NY (US); Michael E. Hogan, Stony Brook, NY (US)

(21) Appl. No.: 16/388,767

(22) Filed: Apr. 18, 2019

Related U.S. Application Data
(60) Provisional application No. 62/660,158, filed on Apr. 19, 2018.
(60) Provisional application No. 62/684,142, filed on Jun. 12, 2018.
(60) Provisional application No. 62/722,704, filed on Aug. 24, 2018.
(60) Provisional application No. 62/788,622, filed on Jan. 4, 2019.

(51) Int. Cl. A61K 35/17; C12N 5/0781; C12N 5/0783; C12Q 1/686; A61P 35/00

(52) U.S. Cl. CPC A61K 35/17; C12N 5/0635; A61P 35/00; C12N 5/0646; C12Q 1/686; C12N 5/0636

(57) ABSTRACT

The present inventions provides systems and methods to manufacture genetically modified lymphocyte cells via the use of linear DNA expression amplicons, and uses of such genetically modified lymphocyte cells to treat disease. The present invention also provides for the composition of genetically modified lymphocyte cells.

### CROSS-REFERENCE TO RELATED APPLICATIONS

This application claims benefit of U.S. Provisional Patent Application No. 62/660,158, filed Apr. 19, 2018, U.S. Provisional Patent Application No. 62/684,142, filed Jun. 12, 2018, U.S. Provisional Patent Application No. 62/722,704, filed Aug. 24, 2018 and U.S. Provisional Patent Application No. 62,788,622, filed Jan. 4, 2019, all of which are hereby incorporated by reference in their entireties.

### TECHNICAL FIELD

The present invention relates to the field of adoptive cell therapy and nucleic acid-based therapies. More specifically, the present invention relates to the manufacture and composition of genetically engineered lymphocyte cells to treat disease.

### BACKGROUND

The genetic engineering of lymphocyte cells to treat disease has gained widespread acceptance. A large number of genetically engineered lymphocyte-based therapies has entered the clinic and several genetic engineered lymphocyte-based therapies have gained regulatory approval for cancer indications. In addition to cancer, there is interest in utilizing engineered lymphocyte-based therapies to treat a wide range of disease indications based on the in vivo production of antibodies.

Adoptive T-cell therapy, currently comprised of chimeric antigen receptor T-cell therapy (“CAR T-cell therapy”) and T-cell receptor therapy (TCR T-cell therapy), is one of the most promising uses of genetically engineered lymphocytes. CAR T-cell therapy has been shown to be highly efficacious against the majority of B-cell malignancies; and many clinical trials are now underway for CAR T-cell therapies directed at other forms of cancer, including non-solid and solid-tumors indications. In CAR T-cell therapy, a population of T-cells is genetically modified to seek target tumor-associated antigens (TAAs) and/or tumor-specific antigens (TSAs) through the introduction of an engineered DNA or RNA based expression vector (gene) encoding an artificial chimeric T-cell receptor specific to one or more TAAs or TSAs. TCR T-cell therapy has also proved promising for the treatment of both cancer and viral infections. In TCR T-cell therapy, an engineered DNA or RNA based expression vector encoding a natural and/or artificial T-cell receptor is transfected into a population of T-cells. The modified T-cell then expresses the T-cell receptor encoded by the expression vector, said T-cell receptor configured to specifically recognize specific tumor cell surface antigen-HLA/MHC class I complexes. In some use cases, CAR T-cell therapy and TCR T-cell therapy can be used in conjunction.

Another promising use of genetically engineered lymphocyte cells is the in vivo production of personalized and/or non-personalized antibodies that confer greater efficacy, longevity and a reduced cost when compared to monoclonal antibodies (mAbs) produced commercially via ex vivo or in vitro means. Such in vivo production of personalized and/or non-personalized antibodies may also be used in conjunction with CAR T-cell and/or TCR T-cell therapies.

Targeted, but non-personalized monoclonal antibodies (mAbs) represent one of the most important medical therapeutic advances of the last 35 years. This type of immune-based therapy is now used routinely against a host of autoimmune diseases, treatment of cancer, as well as infectious diseases. The clinical impact of mAb therapy is impressive, despite its non-personalized nature and challenging production methodologies. However, issues remain that limit the use and dissemination of the current non-personalized mAbs therapeutic approach. Some of these include the high cost of production of these complex biologics that can limit their use in the broader population, particularly in the developing world where they could have a great impact. Furthermore, the frequent requirement for repeat administrations of the mAbs to attain and maintain efficacy can be an impediment in terms of logistics and subject compliance. Additionally, the long-term stability of current mAbs formulations is often short and less than optimal, requiring repeat administration. Subject rejection of conventional non-personalized mAbs is also a problem, as is integration of mAbs with other treatments such as CAR T-cell and/or TCR T-cell therapies.

Heretofore, the process of creating genetically engineered lymphocyte cells has been extremely time consuming, using disparate apparatuses often located in various locations. In addition, one of the major rate-limiting factors in current processes is the time to produce and amplify the synthetic DNA constructs needed to transfect the target lymphocyte cells. At present, this process is undertaken with bacterial plasmids, small circular episomal DNA molecules that can replicate independently of the bacterial chromosomal DNA. In addition to long amplification times, measured in days or weeks, the amplification of DNA constructs via bacterial plasmids has additional drawbacks such as the necessity of complex and expensive purification steps, the risk of endotoxin contamination and challenges with integration into robotic and/or automated workflows.

Moreover, to date, clinical ex vivo transfection of lymphocyte cells has been accomplished only via retro-viruses and/or transposons, each of which carry additional risks above those associated with plasmid DNA. These risks and drawbacks are well documented and include carcinogenesis, immunogenicity, recombination events, broad tropism, limited DNA packaging capacity, long term expression, and difficulty and high costs of production.

Thus, there is a need for genetically engineered lymphocyte cells manufactured without the use of plasmids and/or viral vectors. The use of systems and methods utilizing the polymerase chain reaction (PCR) to create error-free and/or error-mitigated linear DNA expression amplicons for the genetic engineering of lymphocyte cells addresses this need.

### SUMMARY OF THE INVENTION

The present inventions provides systems and methods to manufacture genetically engineered lymphocyte cells via the use of linear DNA expression amplicons, and uses of such genetically engineered lymphocyte cells to treat disease. The present invention also provides for the composition of genetically engineered lymphocyte cells.

In one aspect, the invention provides a method of manufacturing genetically engineered lymphocyte cells, said method comprising: (a) providing a plurality of lymphocyte cells; (b) assembling a DNA expression amplicon template expressing a desired CAR, TCR and/or antibody; (c) amplifying the linear DNA expression amplicon template via PCR to create a plurality of linear DNA expression amplicons; (d) optionally, verifying the sequence of the plurality of linear DNA expression amplicons via NGS; (e) transfecting the plurality of lymphocyte cells with the plurality of linear DNA expression amplicons; (f) optionally, verifying the RNA expressed by the transfected lymphocyte cells via NGS; and (g) optionally, verifying the antibody, TCR and/or CAR structure expressed by the transfected lymphocyte cells via mass spectrometry.

In some embodiments, the plurality of lymphocyte cells can be autologous, allogeneic or a combination of the two.

In one aspect, the invention provides a method of manufacturing genetically engineered lymphocyte cells, said method comprising: (a) providing a plurality of lymphocyte cells; (b) assembling a DNA expression amplicon template expressing a desired CAR, TCR and/or antibody; (c) amplifying the linear DNA expression amplicon template via PCR to create a plurality of linear DNA expression amplicons; (d) optionally, confirming the DNA sequence of a partial quantity of the linear DNA expression amplicons via NGS; (e) optionally, performing in vitro transcription on a partial quantity of the linear DNA expression amplicons to produce RNA and verifying the sequence of the RNA via NGS; (f) optionally, performing in vitro translation of the RNA produced via in vitro transcription to produce a CAR, TCR and/or antibody protein and verifying the produced CAR, TCR and/or antibody protein structure via mass spectrometry; and (g) transfecting the plurality of lymphocyte cells with the remaining quantity of linear DNA expression amplicons.

In another aspect, the invention provides a system for the manufacture of genetically engineered lymphocyte cells, said system comprised of: (a) an input of lymphocytes; (b) a sorting apparatus; (c) a next generation sequencing (NGS) device; (d) a gene synthesizer; (e) a PCR device; (f) a culture storage and expansion device; and (g) a transfection device, wherein all aspects of the system are integrated by robotic process automation. The input of lymphocytes cells can be autologous, allogeneic or a combination of the two. In one embodiment, the lymphocyte cells are B cells. The PCR device may be configured to run large-scale PCR.

In another aspect, the invention provides a system for the manufacture of transfected lymphocyte cells, said system comprised of: (a) an input of lymphocyte cells; (b) a next generation sequencing (NGS) device; (c) a PCR device; (d) optionally, a mass spectrometry device; and (e) a transfection device, wherein all aspects of the system are integrated by robotic process automation. The input of lymphocyte cells can be autologous, allogeneic or a combination of the two. In one embodiment, the lymphocyte cells are T cells or natural killer (NK) cells. The PCR device may be configured to run large-scale PCR.

In another aspect, the invention provides a method of manufacturing genetically engineered B cells that express subject-specific antibodies, said method comprising the steps of: (a) obtaining a sample of a subject's blood; (b) isolating the subject's CD138+ and/or CD38+B-cells; (c) ascertaining the RNA sequence of the antibodies expressed by the subject's CD138+ and/or CD38+B-cells; (d) assembling one or more linear DNA open reading frames (ORF) constructs coding for the expressed antibodies; (e) amplifying and modifying the linear DNA ORF constructs via polymerase chain reaction (PCR) to make linear DNA expression amplicons; (f) verifying the DNA sequence of the linear DNA expression amplicons via NGS; (g) transfecting the isolated CD138+ and/or CD38+B-cells with the verified linear DNA expression amplicons; (h) optionally, verifying the RNA expressed by the transfected CD138+ and/or CD38+B-cells via NGS; (i) optionally, verifying the antibody produced by the transfected CD138+ and/or CD38+B-cells via mass spectrometry; and (j) pooling the verified transfected CD138+ and/or CD38+B-cells.

In another aspect, the invention provides a method of manufacturing genetically engineered B cells that express subject-specific antibodies, said method comprising the steps of: (a) obtaining a sample of a subject's blood; (b) isolating the subject's CD138+ and/or CD38+B-cells; (c) ascertaining the RNA sequence of the antibodies expressed by the subject's CD138+ and/or CD38+B-cells; (d) assembling linear DNA expression amplicon template containing an expression vector coding for the expressed antibodies; (e) amplifying the linear DNA expression amplicon template to make a plurality of linear DNA expression amplicons; (f) verifying the DNA sequence of the linear DNA expression amplicons via NGS; (g) transfecting the isolated CD138+ and/or CD38+B-cells with the verified linear DNA expression amplicons; (h) optionally, verifying the RNA expressed by the transfected CD138+ and/or CD38+B-cells via NGS; (i) optionally, verifying the antibody produced by the transfected CD138+ and/or CD38+B-cells via mass spectrometry; and (j) pooling the verified transfected CD138+ and/or CD38+B-cells.

In alternative embodiments, the invention provides a method of treating a subject with genetically engineered B-cells that express subject-specific antibodies, said method comprising the steps of: (a) obtaining a sample of a subject's blood; (b) isolating the subject's CD138+ and/or CD38+B-cells; (c) ascertaining the RNA sequence of the antibodies expressed by the subject's CD138+ and/or CD38+B-cells; (d) assembling a DNA expression amplicon template expressing the desired antibodies; (e) amplifying the linear DNA expression amplicon template via PCR to produce a plurality of linear DNA expression amplicons; (f) optionally, verifying the DNA sequence of the linear DNA expression amplicons via NGS; (g) transfecting the subject's B-cells with the verified linear DNA expression amplicons; (h) optionally, verifying the RNA expressed by the transfected B-cells via NGS; (i) optionally, verifying the structure of the antibody produced by the transfected B-cells via mass spectrometry; (j) pooling the transfected B-cells; and (k) introducing the pooled transfected B-cells to a subject. The transfected B-cells may be autologous, allogeneic or a combination of the two.

In another aspect, the invention provides a plurality of genetically engineered lymphocyte cells manufactured by the process comprising the steps of: (a) providing and input of lymphocyte cells; (b) assembling one or more linear DNA ORF constructs; (c) amplifying and modifying the linear DNA ORF constructs via PCR to make linear DNA expression amplicons; (d) optionally, verifying the sequence of the linear DNA expression amplicons via NGS; (e) transfecting the lymphocyte cells with the linear DNA expression amplicons; (f) optionally, verifying the RNA expressed by the transfected lymphocyte cells via NGS; and (g) optionally, verifying the structure of the antibody, TCR and/or CAR expressed by the transfected lymphocyte cells via mass spectrometry. The transfected lymphocyte cells may be CD138+ or CD38+B-cells, T cells and/or a NK cells, and may be autologous, allogeneic or a combination of the two.

In another aspect, the invention provides for an isolated lymphocyte cell comprising a linear DNA expression amplicon, wherein said amplicon include an expression cassette for a CAR, TCR and/or antibody. In an embodiment, the isolated lymphocyte cell may be a B cell, T cell or NK cell. In an embodiment, the isolated lymphocyte may be autologous, allogeneic or a combination of the two. In one embodiment, the lymphocyte cell contains a linear DNA expression amplicon that is configured for episomal nuclear persistence and/or episomal nuclear replication. The linear DNA expression amplicon may be manufactured via large-scale PCR.

In another aspect, the invention provides for a plurality of transfected lymphocyte cells manufactured by the process comprising the steps of: (a) providing an input of lymphocyte cells; (b) assembling a DNA expression amplicon template; (c) amplifying the linear DNA expression amplicon template via PCR to create a plurality of linear DNA expression amplicons; (d) optionally, verifying the sequence of the plurality of linear DNA expression amplicons via NGS; (e) transfecting the lymphocyte cells with the plurality of linear DNA expression amplicons; (f) optionally, verifying the RNA expressed by the transfected lymphocyte cells via NGS; and (g) optionally, verifying the structure of the antibody, TCR and/or CAR expressed by the transfected lymphocyte cells via mass spectrometry. The transfected lymphocyte cells can be autologous, allogeneic or a combination of the two. The PCR amplification of the linear DNA expression amplicons can be accomplished via large-scale PCR.

In another aspect, the invention provides pharmaceutical composition for use in treating a disorder, wherein the composition comprises a lymphocyte cell containing a linear DNA expression amplicon expressing an antibody, TCR, and/or CAR. In some embodiments, the lymphocyte cell is a CD138+ or CD38+B-cell, a T cell and/or a NK cell. In some embodiments, the linear DNA expression amplicon may be produced via large-scale PCR. In one embodiment, the lymphocyte cell contains a linear DNA expression amplicon that is configured for episomal nuclear persistence and/or episomal nuclear replication.

In another aspect, the invention provides a method of treating a disorder in a subject, wherein the method comprises (a) identifying a TAA, TSA, neoepitope, other epitope, mutational associated neoantigens, oncoviral, oncofetal, lineage-restricted, and/or over-expressed tumor antigens of therapeutic relevance to a subject; (b) providing a plurality of lymphocyte cells; (c) assembling a linear DNA expression amplicon template containing an expression cassette encoding a TCR, CAR and/or antibody against the identified TAA, TSA, neoepitope, other epitope, mutational associated neoantigens, oncoviral, oncofetal, lineage-restricted, and/or over-expressed tumor antigens; (d) amplifying the linear DNA expression amplicon template via PCR to create a plurality of linear DNA expression amplicons; (e) optionally, verifying the sequence of the plurality of linear DNA expression amplicons via NGS; (f) transfecting the lymphocyte cells with the plurality of linear DNA expression amplicons; (g) optionally, verifying the RNA expressed by the transfected lymphocyte cells via NGS; and (h) optionally, verifying the structure of the antibody, TCR and/or CAR expressed by the transfected lymphocyte cells via mass spectrometry; and (i) administering the transfected lymphocyte cells to a subject. The transfected lymphocyte cells may be autologous, allogeneic or a combination of the two. In some embodiments, the linear DNA expression amplicon may be produced via large-scale PCR. In an embodiment, the linear DNA expression amplicon is configured for episomal nuclear persistence and/or episomal nuclear replication.

In another aspect, the invention provides a method of treating a disorder in a subject, wherein the method comprises (a) providing a plurality of lymphocyte cells; (b) assembling a linear DNA expression amplicon template; (c) amplifying the linear DNA expression amplicon template via PCR to create a plurality of linear DNA expression amplicons; (d) optionally, verifying the sequence of the plurality of linear DNA expression amplicons via NGS; (e) transfecting the lymphocyte cells with the plurality of linear DNA expression amplicons; (f) optionally, verifying the RNA expressed by the transfected lymphocyte cells via NGS; and (g) optionally, verifying the structure of the antibody, TCR and/or CAR expressed by the transfected lymphocyte cells via mass spectrometry; and (h) administering the transfected lymphocyte cells to a subject.

In another aspect, the invention provides a method of treating a disorder in a subject, wherein the method comprises (a) obtaining a sample of a subject's blood; (b) isolating the subject's CD138+ and/or CD38+B-cells; (c) ascertaining the RNA sequence of the antibodies expressed by the subject's CD138+ and/or CD38+B-cells; (d) assembling a DNA expression amplicon template expressing the desired antibodies; (e) amplifying the linear DNA expression amplicon template via PCR to produce a plurality of linear DNA expression amplicons; (f) optionally, verifying the DNA sequence of the linear DNA expression amplicons via NGS; (g) transfecting the isolated CD138+ and/or CD38+B-cells with the verified linear DNA expression amplicons; (h) optionally, verifying the RNA expressed by the transfected CD138+ and/or CD38+B-cells via NGS; (i) optionally, verifying the structure of the antibody produced by the transfected CD138+ and/or CD38+B-cells via mass spectrometry; (j) pooling the transfected CD138+ and/or CD38+B-cells; and (k) administering the pooled transfected CD138+ and/or CD38+B-cells cells to the subject.

In another aspect, a method of treating cancer is provided, said method comprising the steps of administering to a subject in need thereof, an engineered lymphocyte cell comprising a linear DNA expression amplicon, wherein said amplicon includes an expression cassette for a CAR, TCR and/or antibody.

In some embodiments of the disclosed systems, methods and compositions, the amplification of the linear DNA expression amplicon template via PCR to create a plurality of linear DNA expression amplicons is accomplished via large-scale PCR.

In some embodiments of the disclosed systems, methods and compositions, the linear DNA expression amplicons may be configured for episomal nuclear persistence and/or episomal nuclear replication.

### BRIEF DESCRIPTION OF THE FIGURES

FIG. 1 illustrates the components of one embodiment of the system.

FIG. 2 illustrates the design of one embodiment of a linear DNA expression amplicon.

FIG. 3 is a flow diagram of one embodiment of a method of producing genetically engineered lymphocytes via linear DNA amplicons.

FIG. 4 is a diagram of one embodiment of a linear DNA expression amplicon configured for episomal nuclear persistence and/or episomal nuclear replication.

FIG. 5 is a flow diagram of one embodiment of a method of assembling a linear DNA expression amplicon configured for episomal nuclear persistence and/or episomal nuclear replication.

FIG. 6 is a diagram of an embodiment of a linear DNA expression amplicon expressing a CAR configured for episomal nuclear persistence and/or episomal nuclear replication.

FIG. 7 is a flow diagram of one method of manufacturing genetically engineered lymphocyte cells via linear DNA expression amplicons.

FIG. 8 is a diagram of one embodiment of a linear DNA expression amplicon template encoding green fluorescent protein (GFP) as derived from a plasmid.

FIG. 9 is a chart showing expression and cell viability data from human T cells transfected with a linear DNA expression amplicon encoding for GFP.

FIG. 10 is a line graph showing GFP expression level for various linear DNA expression amplicon concentrations transfected into human T cells.

FIG. 11 is a diagram of one embodiment of a linear DNA expression amplicon template encoding a CD19 CAR as derived from a plasmid.

FIG. 12 is a flow diagram of one method of manufacturing genetically engineered lymphocyte cells via linear DNA expression amplicons.

### DETAILED DESCRIPTION

The present invention provides systems and methods for the manufacture of genetically engineered lymphocyte cells via the use of linear DNA expression amplicons and uses of such genetically engineered lymphocyte cell compositions to treat disease in a subject. The present invention also provides for the composition of genetically engineered lymphocyte cells, said genetically engineered lymphocyte cells expressing an antibody, a TCR and/or a CAR from a transfected linear DNA expression amplicon. Also provided is a system and method of making personalized subject-specific genetically modified lymphocyte cells via the use of linear DNA expression amplicons.

### Definitions

The term “amplicon” as used herein means a piece of DNA or RNA that is the product of an enzymatic or chemical based amplification event or reaction. Amplification events or reactions include, without limitation, the polymerase chain reaction (PCR), loop mediated isothermal amplification, rolling circle amplification, nucleic acid sequence base amplification, and ligase chain reaction or recombinase polymerase amplification. An amplicon may be comprised of single stranded and/or double stranded DNA, and/or a combination thereof.

The term “autologous” means that cells, a cell line, or a cell population used for treating a subject originated from said subject.

The term “allogeneic” means cells, a cell line, or a cell population used for treating a subject did not originate from said subject and are derived from a third-party donor.

The term “antibody” (Ab) includes, without limitation, immunoglobulin, which binds specifically to a target (antigen). In general, an antibody can comprise at least two heavy chains and two light chains interconnected by disulfide bonds, or an antigen-binding portion thereof. Each heavy chain comprises a heavy chain variable region and a heavy chain constant region. The heavy chain constant region comprises three constant domains, CH1, CH2 and CH3. Each light chain comprises a light chain variable region and a light chain constant region. The variable regions of the heavy and light chains contain a binding domain that interacts with target/antigen. This also includes modified and/or truncated derivatives of the antibody motif, including those manufactured by chemical synthesis.

The term “assembling” or “assemble” in relation to DNA means the creation of a DNA sequence via artificial gene synthesis which may include photolithographic means, oligonucleotide synthesis, solid-phase DNA synthesis or any other means of gene synthesis know in the art capable of producing DNA sequences of the necessary length and fidelity. The term “assembling” or “assemble” in relation to DNA could also mean deriving a DNA sequence from a plasmid via methods known in the art, including but not limited to molecular cloning or PCR based methods.

The term “cancer” means any disease caused by an uncontrolled division and/or proliferation of abnormal cells in any part of a subject's body.

The term “continuous flow PCR device” means a PCR device as disclosed in U.S. Pat. Nos. 8,293,471; 8,986,982; and 8,163,489.

The term “episomal” means a piece of DNA that replicates independently from chromosomal DNA. Episomal DNA may reside in a cell's nucleus.

As used herein, the term “expression” refers to the transcription and/or translation of an expression cassette or other aspect of a linear DNA expression amplicon.

The term “expression cassette” means a DNA sequence consisting of one or more genes and the sequences controlling their expression. At a minimum, an expression cassette shall include a promoter (or other expression control sequence) and an open reading frame (ORF).

The term “expression control sequence” means a nucleic acid sequence that directs transcription of a nucleic acid and/or open reading frame. An expression control sequence can be a promotor or an enhancer.

The term “linear DNA ORF construct” means a piece of linear DNA containing the open reading frames (ORF) for, inter alia, the heavy and light chains of a desired antibody, a CAR and/or TCR.

The term “linear DNA expression amplicon” means a linear DNA amplicon comprising a linear DNA ORF construct and/or expression cassette, as well as other additional DNA sequences necessary and/or advantageous for expression of the desired ORF, expression cassette, gene, DNA sequence, antibody, antigen receptor, CAR, and/or TCR in a lymphocyte cell. Such other additional DNA constructs may include, without limitation, expression control sequences and configurations for episomal nuclear persistence and/or episomal nuclear replicaiton. As used herein, linear DNA expression amplicons are produced by enzymatic and/or chemical based amplification and modification methodologies, including without limitation, PCR, loop mediated isothermal amplification, rolling circle amplification, nucleic acid sequence base amplification, photolithography assembly, and ligase chain reaction or recombinase polymerase amplification. A linear DNA expression amplicon can be produced by large-scale PCR. A linear DNA expression amplicon can be single or double stranded.

The term “lymphocyte” as used herein includes agranulocytes, without limitation, natural killer (NK) cells, CTLs, T cells and B cells. The term lymphocyte also includes all other types of immune cells of hematopoietic origin functionally involved in the initiation and/or execution of innate and/or adaptive immune responses.

The term a “subject” is any mammal, including without limitation humans, monkeys, farm animals, pets, horses, dogs and cats. In an exemplary embodiment, the subject in human.

The term “next generation sequencing” (NGS) includes any form of high-throughput DNA or RNA sequencing. This includes, without limitation, sequencing by synthesis, sequencing by ligation, nanopore sequencing, single-molecule real-time sequencing and ion semiconductor sequencing.

The term “promoter” refers to a DNA sequence capable of controlling the expression of a ORF, linear DNA ORF construct, expression cassette, or a functional RNA.

The term “transfection” means the uptake of exogenous or heterologous RNA or DNA by a cell. Without limitation, transfection may be accomplished by direct uptake, electroporation, chemical or other substance based methods (e.g. calcium chloride, rubidium chloride, alcohol, DEAE-dextran) lipofection, soluporation, cationic liposomes, cationic polymers, lipoplexes, synthetic branched dendrimers, microprojectile bombardment, cellular surgery and/or viral transduction.

The term “large-scale PCR” means a PCR reaction wherein the total PCR reaction volume is greater than 0.7 liters. Large-scale PCR may be performed in a single reaction vessel or may be performed in a plurality of reaction vessels simultaneously.

The term “CAR” means a chimeric antigen receptor which is a recombinant biomolecule that contains an extracellular recognition domain, a transmembrane region, and an intracellular signaling domain. The extracellular recognition domain comprises a recognition element that specifically binds to a molecule present on the cell surface of a target cell. The transmembrane region anchors the CAR in the membrane. The intracellular signaling domain comprises the signaling domain and optionally comprises one or more co-stimulatory signaling domains. The extracellular recognition domain may bind a tumor associated antigen (TAA), a tumor specific antigen (TSA) or any other target molecule. The term CAR also includes T cells redirected for universal cytokine-mediated killing (TRUCKs).

The term “TCR” means T-cell receptor, which is a molecule found on the surface of a Tcell, that is responsible for recognizing fragments of antigen as peptides bound to MHC molecules. The TCR may recognize a TAA, TSA or any other target molecule.

The use of the alternative (e.g., “or”) should be understood to mean either one, both, or any combination thereof of the alternatives. As used herein, the indefinite articles “a” or “an” should be understood to refer to “one or more” of any recited or enumerated component.

Linear DNA Expression Amplicon

A linear DNA expression amplicon can be a linear DNA amplicon comprising a linear DNA ORF construct and other additional DNA constructs necessary for expression of the desired gene or DNA sequence, including without limitation an antibody, antigen receptor, CAR, and/or TCR in a lymphocyte cell. A linear DNA expression amplicon can also be a linear DNA amplicon comprising one or more expression cassettes and other DNA constructs. Such other additional DNA constructs may include, without limitation, expression control sequences, configurations for episomal nuclear persistence/replication and exonuclease degradation protections. A linear DNA expression amplicon can also be comprised of RNA and/or sequence corresponding to RNA. A linear DNA expression amplicon can be single or double stranded, preferably double stranded, and may be of any base pair length. In some embodiments, a linear DNA expression amplicon can be between 1 kb to 20 kb in length. In other embodiments, a linear DNA expression amplicon can be between 3 kb to 25 kb in length. In yet another embodiment, a linear DNA expression amplicon can be between 10 kb to 50 kb in length. In a preferred embodiment, a linear DNA expression amplicon can be between 1 kb to 10 kb in length.

Optionally, a linear DNA expression amplicon may also include one or more enhancers, a T7 promoter, DNA sequences that upon transcription will give rise to a poly-a tail suitable for expression in mammalian cells, an ORF for in-frame fusion tag (fusion ORF), telomeric sequences, a centromere, CPG open reading frames, and/or chemical and/or peptide-based modifications. A linear DNA expression amplicon is translatable by a lymphocyte cell into a desired antigen, antigen receptor, CAR, TCR or other target polypeptide or other therapeutically relevant peptide, polypeptide, protein and/or RNA. An expression cassette contained within a linear DNA expression amplicon may be identical to an expression cassette utilized in a plasmid or other non-amplicon based expression vector, or be modified for use specifically in a linear DNA expression amplicon. Exemplary promoters include without limitation, CMV, T7, EF1a, SV40, PGK1, Ubc, CAG, TRE, UAS and Ac5. Exemplary enhancers include without limitation SV40, CMV enhancer and woodchuck hepatitis virus posttranscriptional regulatory element (WPRE). Exemplary terminators include without limitation SV40 polyadenylation/late polyadenylation signal, bovine growth hormone (bGH) polyadenylation signal and rabbit beta-globin (rbGlob) polyadenylation signal.

A linear DNA expression amplicon may include an in-frame fusion ORF that is translatable into one or more fusion tags such as small ubiquitin-related modifier (SUMO), ubiquitin (Ub), maltose binding protein (MBP), glutathione S-transferase (GST), thioredoxin (TRX), Strep-tag, Strep-tag II and NUS A. A fusion tag is a short peptide, protein domain, or entire protein that can be fused to a target protein. When the ORF and the fusion ORF of a linear DNA expression amplicon are both translated, the target protein with a fusion tag is produced creating a fusion protein.

Through the use of one or more fusion tags, the expression level of a protein, antigen, antigen receptor, antibody, CAR, TCR or other target polypeptide encoded by the linear DNA expression amplicon can be increased or modified. In one embodiment, a linear DNA expression amplicon expressing a CAR and also containing a fusion ORF for a fusion tag may be utilized to provide high level expression of the CAR in a lymphocyte cell. In one exemplary embodiment, the fusion ORF encodes for a human or mammalian SUMO fusion tag. The fusion tag may also be utilized to identify lymphocyte cells that are expressing the desired CAR. The fusion tags may be removed from the target protein, CAR (including a CAR) after expression by means of appropriate cleaving tags. The CAR produced by linear DNA expression amplicons may be designed such that the fusion tag does not affect the efficacy of the expressed CAR, and may be engineered to avoid fusion tag fixation in or near the antigen receptor region and/or the single-chain variable fragment (scFv) region.

A linear DNA expression amplicon can be modified via the incorporation of cell-penetrating peptides (CPPs). CPPs are capable of acting as a powerful transport vector for the intracellular delivery of linear DNA expression amplicons though the cell membrane, and in certain cases, into the cell nucleus. Exemplary CPPs may be hydrophilic, polycationic, amphiphilic or contain a periodic sequence.

Linear DNA expression amplicons can also be modified via the incorporation of peptides containing one or more nuclear localization sequences (NLSs) to provide for efficient transport to the cell nucleus for translation, and to minimize time spent in the cytosol of target cells post transfection. The use of NLS containing peptides is advantageous in both dividing and non-dividing target cells. The NLSs may be monopartite or bipartite, or take the form of other non-classical NLSs. The NLS containing peptides may be complexed with linear DNA expression amplicons via electrostatic interactions during or after PCR amplification. In addition, NLS containing peptides may also be conjugated to linear DNA expression amplicons via random covalent attachment and/or by site specific covalent conjugation. Site specific covalent conjugation may be accomplished via the use of linear DNA expression amplicons with amine modified termini or other modified termini, or through the use of PNA mediated PCR clamping (hybridization) of a PNA linked to a NLS containing peptide. An exemplary form of site specific covalent conjugation is attachments of one or more NLS containing peptides at the 5′ and/or 3′ termini of the linear DNA expression amplicon. Addition of one or more NLS containing peptides to linear DNA expression amplicons may be undertaken during or after PCR amplification. The covalent conjugation of a NLS containing peptide to a linear DNA expression amplicon may occur via the NLS containing peptide's C- or N-terminal such that the NLS containing peptide binding properties to a cell's transport proteins are not affected. NLS containing peptides may also be linked to one or more nanoparticles and then covalently bound to or complexed with linear DNA expression amplicons.

A linear DNA expression amplicon can also include protective features to reduce degradation by exonuclease or other factors. A protective feature can be introduced at the 5′ and/or 3′ termini of the linear DNA expression amplicon. In one embodiment, a length of noncoding DNA sequence extending beyond the expression cassette of a linear DNA expression amplicon is added. This noncoding DNA may be G-quadruplex structures or may be other noncoding DNA of a known sequence and length. G-quadruplex structures may be added to a linear DNA expression amplicon via the use of modified PCR primers. Phosphorothioate-modification can be used to protect linear DNA expression amplicons against degradation. Said phosphorothioate-modification may be accomplished via phosphorothioate modified PCR primers. Peptide nucleic acid (PNA) sequences may also be used to protect the 3′ and/or 5′ termini of amplicon expression vectors.

The template for a linear DNA expression amplicon may be obtained from any suitable source, including without limitation, a plasmid, de novo gene synthesis, de novo gene synthesis, oligonucleotide synthesis, artificial gene synthesis, solid-phase gene synthesis, or one or more DNA amplicons produced via PCR.

Episomal Nuclear Persistence/Episomal Nuclear Replication

A linear DNA expression amplicon can be configured for episomal nuclear persistence and/or episomal nuclear replication through the incorporation of elements of an artificial chromosome, which may include, without limitation, telomeres, centromeres and origins of replication. Episomal nuclear persistence and/or episomal nuclear replication of a linear DNA expression amplicon allows for persistence of the linear DNA expression amplicon in a transfected lymphocyte cell through a number of cell divisions, thus increasing the efficacy of a lymphocyte cell population transfected with linear DNA expression amplicons in a subject.

Linear DNA expression amplicons configured for episomal nuclear persistence and/or replication may include one or more components of an artificial chromosome. An artificial chromosome performs the critical functions of natural chromosomes and may include one or more origins of replication, telomeres, kinetochores and/or centromeres. The artificial chromosomes, or components thereof, may be derived from a yeast artificial chromosome (YAC), a bacterial artificial chromosome (BAC) and/or a mammalian artificial chromosome (MAC), including without limitation a human artificial chromosome (HAC).

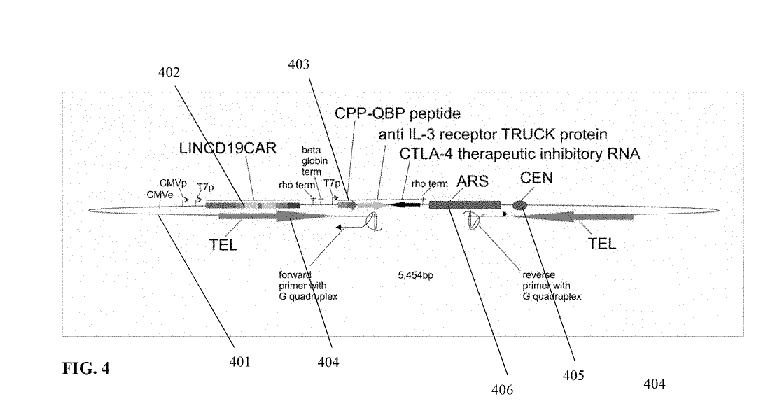

FIG. 4 is a diagram of an exemplary linear DNA expression amplicon configured for episomal nuclear replication and/or episomal nuclear persistence. As shown in FIG. 4, the linear DNA expression amplicon (401) contains an expression cassette for one or more therapeutically relevant polypeptides, protein, antibody, TCR, CAR and/or other amino acid, here an anti-CD19 CAR (402), and one or more CPPs (403), as well as the components of an artificial chromosome, which include in this embodiment, telomere termini (404), a centromere (405) and an origin of replication (406). The origin of replication may be an autonomously replicating sequence (ARS). The ARS may be derived from a yeast genome.

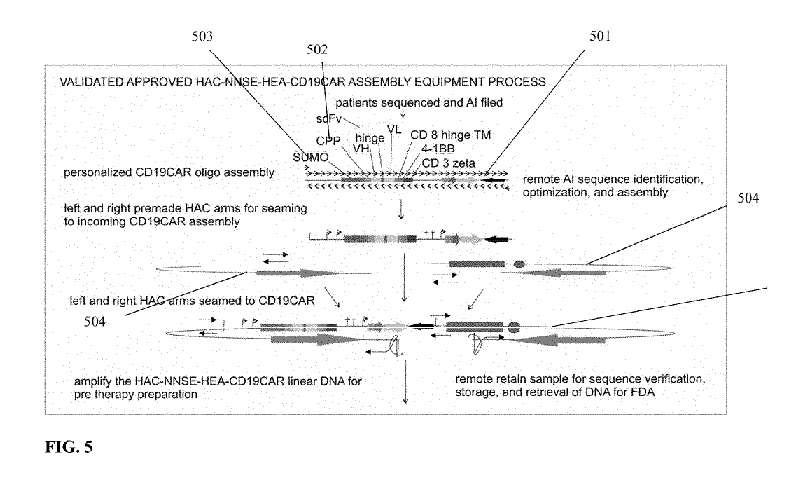

Turning to FIG. 5, one embodiment of a method of assembling a linear DNA expression amplicon configured for episomal nuclear replication and/or episomal nuclear persistence is shown. First, one or more expression cassettes for one or more therapeutically relevant polypeptides, protein, antibody, TCR, CAR and/or other amino acid are chosen and assembled (501). Expression cassettes for in vitro CPP expression (502) and enhancers (503) may also be included. Next, via seaming PCR, pre-manufactured artificial chromosome components (504) are attached to the 3′ and 5′ ends of the chosen expression cassettes. Pre-manufactured artificial chromosome components may be comprised of one or more telomeres, centromeres and/or origins of replication. The pre-manufactured artificial chromosome components may also include G-quadruplex structures on their respective termini. Once the pre-manufactured artificial chromosome components are attached to the 3′ and 5′ ends of the chosen expression cassettes via seaming PCR, a linear DNA expression amplicon configured for episomal nuclear persistence and/or episomal nuclear replication is formed (505). The now formed linear DNA expression amplicon configured for episomal nuclear persistence and/or episomal nuclear replication can be amplified via PCR, large-scale PCR or other form of enzymatic amplification, and transfected into lymphocyte cells.

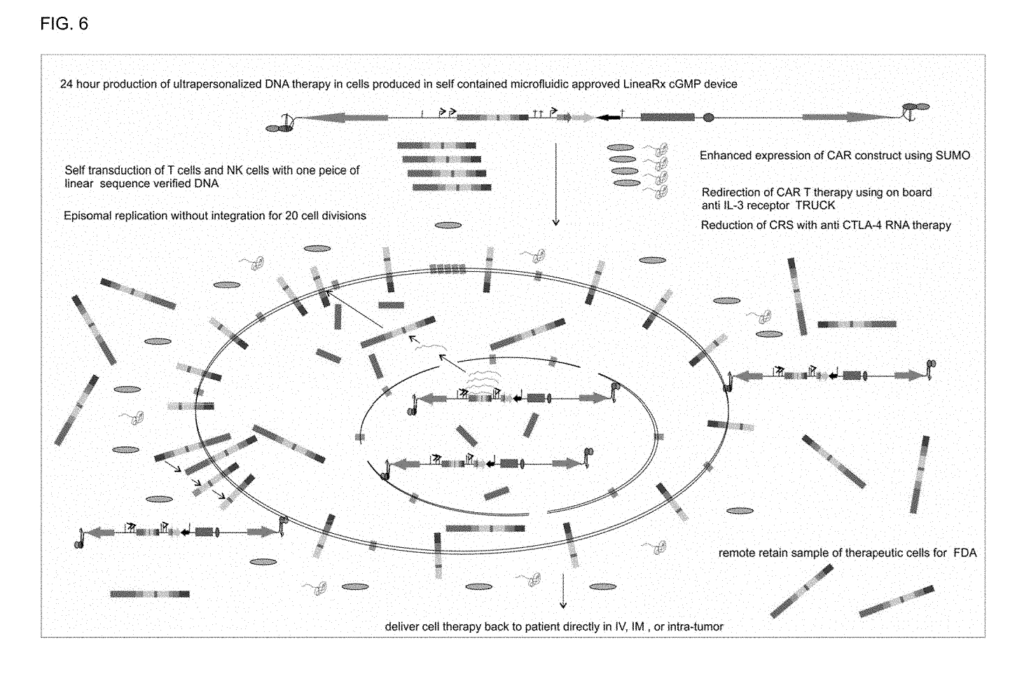

FIG. 6 is a workflow diagram in which one embodiment of a linear DNA expression amplicon configured for episomal nuclear persistence and/or episomal nuclear replication can be utilized in CAR-T cell therapy. The linear DNA expression amplicon configured for episomal nuclear persistence and/or episomal nuclear replication, with CPPs expressed via in an in vitro transcription and translation event and bound to the amplicon expression vector termini via G-quadruplex or other means, and containing an expression cassette for a therapeutic CAR, are transfected into target lymphocytes ex vivo via CPP. The linear DNA expression amplicon configured for episomal nuclear persistence and/or episomal nuclear replication may also be transfected into a target lymphocyte cell via chemical transection, electroporation, soluporation, cell surgery/nano-delivery or any other transfection technology known in the art. Exemplary lymphocytes are T or NK cells. The transfected lymphocytes, now expressing the CAR, are expanded ex vivo. Due to the fact that the transfected linear DNA expression amplicon resident in the cell's nucleus contains the components of an artificial chromosome the linear DNA expression amplicon is able to undergo episomal replication and/or persistence during cell division, without genomic integration, and thus the expanded cell line population also expresses the desired CAR or other desired antigen, antibody or TCR. Episomal persistence and/or replication will subsist until such time as the telomere regions of the linear DNA expression amplicon are degraded or destroyed. The telomere degradation rate of a linear DNA expression amplicon is tunable, such that episomal nuclear persistence and/or episomal nuclear replication through a defined number of cell divisions can be modified.

System and Method for Producing Engineered Lymphocyte Cells via Linear DNA Expression Amplicons

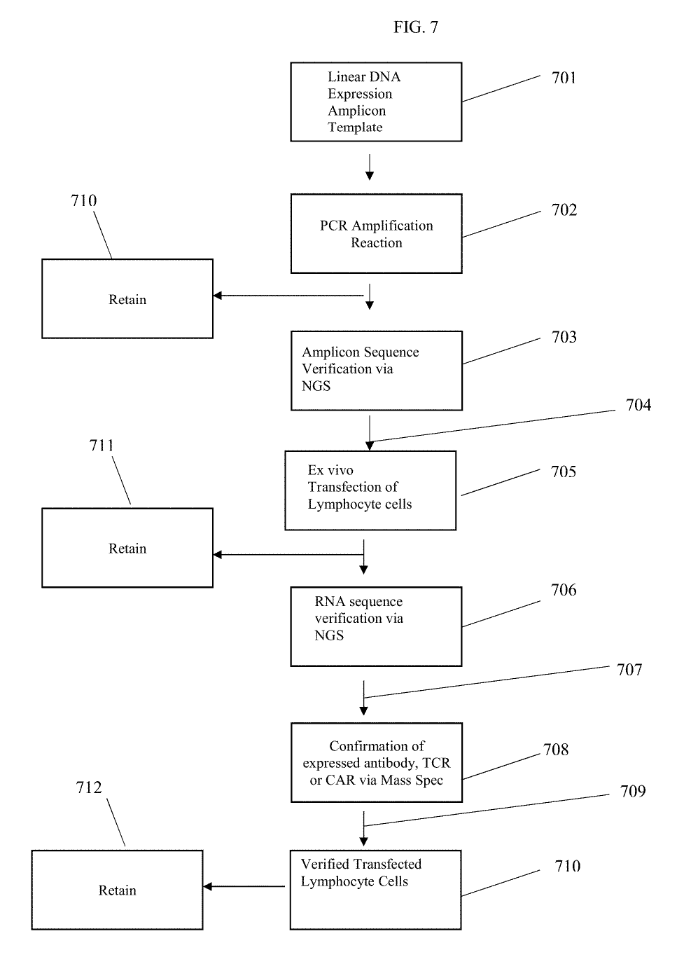

In one aspect, shown in FIG. 7, the invention provides a method of manufacturing genetically modified lymphocyte cells, said method comprising: (a) providing a plurality of lymphocyte cells; (b) assembling a linear DNA expression amplicon template containing an expression cassette for a desired CAR, TCR and/or antibody; (c) amplifying the linear DNA expression amplicon template via PCR to create a plurality of linear DNA expression amplicons; (d) optionally, verifying the sequence of the plurality of linear DNA expression amplicons via NGS; (e) transfecting the plurality of lymphocyte cells with the plurality of linear DNA expression amplicons; (f) optionally, verifying the RNA expressed by the transfected lymphocyte cells via NGS; and (g) optionally, verifying the antibody, TCR and/or CAR expressed by the transfected lymphocyte cells via mass spectrometry.

The plurality of lymphocyte cells may be isolated by any means known in the art, including flow cytometry, or a population of lymphocyte cells may be provided. The plurality of lymphocyte cells can be allogeneic or autologous, or a combination of the two. If allogeneic, lymphocyte cells may be modified to reduce donor incompatibility, graft versus house disease and/or host versus graft rejection. Allogeneic lymphocyte cells may be genetically engineered to down regulate major histocompatibility class I (MHC class I) cell surface expression. In addition to a CAR, TCR and/or antibody expression cassette, a linear DNA expression amplicon may also contain an expression cassette encoding for a protein which will down regulate MHC class I cell surface expression in a lymphocyte cell; or the ORF for a protein which will down regulate MHC class I cell surface expression may be contained on the same expression cassette as a CAR, TCR and/or antibody.

The linear DNA expression amplicon template (701) may be manufactured/assembled by gene or oligonucleotide synthesis, obtained from a plasmid or may be a preexisting linear DNA expression amplicon. If obtained from a plasmid, the expression cassette for the desired CAR, TCR, antibody or other therapeutic peptide can be excised from the plasmid via methods known in the art and converted to a linear DNA expression amplicon template. The expression cassette excised from the plasmid may be used unmodified as a linear DNA expression amplicon template or it may be modified before use as a linear DNA expression amplicon template. Such modifications may include protection against exonuclease degradation, additional or alternative expression control sequences, or configuration for episomal nuclear persistence and/or episomal nuclear replication.

The linear DNA expression amplicon template is then amplified via PCR (702). Exemplary embodiments utilize extremely high fidelity polymerase such as Q5® polymerase (NEB Biolabs, Inc. USA) with an error rate less than 5.3×10⁻⁷ in the PCR reaction. PCR conditions can also be optimized to increase fidelity. Two step-PCR can be used. Large-scale PCR can be used in conjunction with high fidelity polymerase to amplify the linear DNA expression amplicon template to create a high copy number of linear DNA expression amplicons. In an exemplary embodiment, a continuous flow PCR device is used for linear DNA expression amplicon template amplification. The continuous flow PCR device may be large-scale PCR.

After PCR amplification, the linear DNA expression amplicons may be sequence verified via NGS (703). In one embodiment, PCR amplification is accomplished in a device which contains a plurality of separate reaction vessels. The sequences of the linear DNA expression amplicons in each separate reaction vessels can be verified via a NGS apparatus. The reaction vessels containing the lowest or non-existence error rates as quantified by NGS can be pooled (704) to create a large copy number of NGS sequence confirmed linear DNA expression amplicons, thus mitigating the already extremely low error rate of the high fidelity polymerase used in the PCR reaction. The plurality of lymphocyte cells are then transfected with the pooled sequence confirmed linear DNA expression amplicons (705).

Transfection of the lymphocyte cell population (705) can be accomplished via any transfection methodology known in the art. Exemplary methods include, without limitation, direct uptake, electroporation, chemical or other substance based methods (e.g. calcium chloride, rubidium chloride, alcohol, DEAE-dextran) lipofection, cationic liposomes, soluporation, cationic polymers, lipoplexes, synthetic branched dendrimers, microprojectile bombardment and cellular surgery. Viral transduction or a transposon/transposase systems can also be used.

Post-transfection, the transfected lymphocyte cell population may undergo RNA sequence analysis (706) via NGS to ascertain whether the transfected cells are expressing the correct RNA sequence based on the desired CAR, TCR and/or antibody to be expressed. Successful transfection may be confirmed via GFP or eGFP analysis. An expression cassette for GFP and/or eGFP may be included as part of a linear DNA expression amplicon. This additional optional step of RNA sequence analysis via NGS further mitigates against PCR amplification error by confirming proper RNA expression for the desired TCR, CAR or antibody by one or more transfected cells. Transfection of the lymphocyte cell population can be undertaken in separate reaction vessels, or each transfected lymphocyte cell can be sorted and placed into its own discrete vessel. RNA sequence verified transfected lymphocyte cells can be pooled (707) for further analysis or administration to a subject, while any transfected cells expressing improper RNA sequences can be discarded.

The transfected lymphocyte cell population, which may have been RNA sequence verified, can also undergo additional verification of the structure of the expressed TCR, CAR and/or antibody via mass spectrometry (708). The analysis of cell expression via mass spectrometry, so called high-throughput proteomics (HTP), is capable of accurately analyzing the proteins expressed by a large number of cells, including TCRs, CARs and/or antibodies. HTP based on electrospray ionization, matrix-assisted desorption/ionization, a combination of these two techniques, or other techniques can be used. Transfected lymphocyte cells with confirmed expression of proper TCR, CAR and/or antibody structure can be pooled (709). The pooled transfected lymphocyte cells (710), which now may have undergone DNA sequence confirmation, RNA sequence confirmation and expressed TCR, CAR and/or antibody structure confirmation, can now be delivered to the subject. Retains for regulatory compliance (711 and 712) can be obtained and preserved.

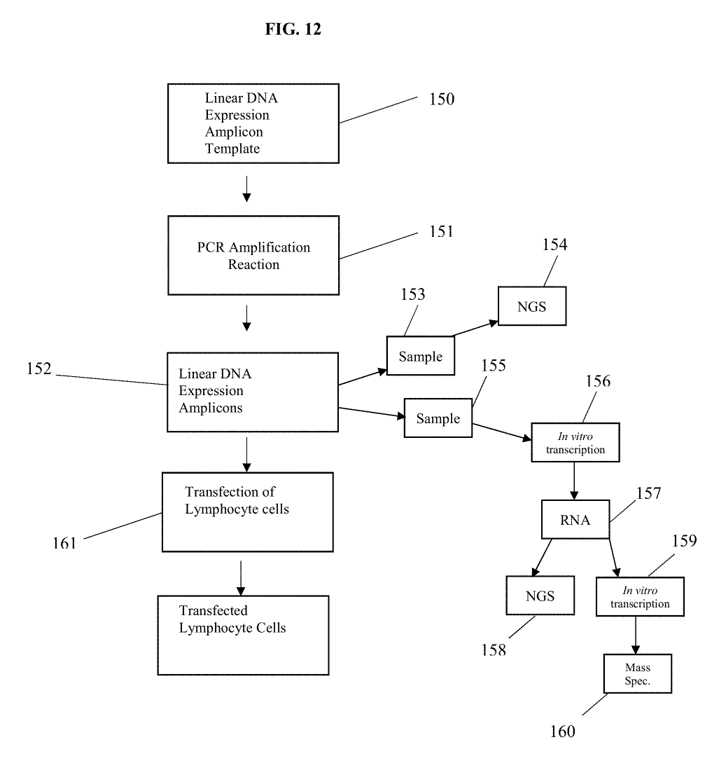

In an alternative embodiment, shown in FIG. 12, an alternative method of manufacturing genetically engineered lymphocyte cells is disclosed. This method comprises: (a) providing a plurality of lymphocyte cells; (b) assembling a DNA expression amplicon template expressing a desired CAR, TCR and/or antibody (150); (c) amplifying the linear DNA expression amplicon template via PCR (151) to create a plurality of linear DNA expression amplicons (152); (d) optionally, confirming the DNA sequence of a quantity (153) of the linear DNA expression amplicons via NGS (154); (e) optionally, performing in vitro transcription (156) on a quantity of the linear DNA expression amplicons (155) to produce RNA (157) and verifying the sequence of the RNA via NGS (158); (f) optionally, performing in vitro translation (159) of the RNA produced via in vivo transcription to produce a CAR, TCR and/or antibody protein and verifying the protein structure via mass spectrometry (160); and (g) transfecting the plurality of lymphocyte cells with the remaining quantity of linear DNA expression amplicons (161).

In vitro transcription and/or translation may be accomplished via any known means, including the use of commercially available cell-free expression system and/or cell-free protein synthesis systems. These systems may be derived from, without limitation, E. coli (ECE), rabbit reticulocytes (RRL), wheat germ (WGE) and/or insect cells (ICE). A linear DNA expression amplicon can be modified to facilitate in vitro transcription and/or translation.

In another aspect, a method of manufacturing personalized genetically engineered lymphocyte cells is disclosed, said method comprising: (a) ascertaining a specific TAA, TSA, neoepitope, other epitope, neoantigen, mutational associated neoantigens, oncoviral, oncofetal, lineage-restricted, and/or over-expressed tumor antigens of therapeutic relevance to a subject; (b) providing a population of lymphocyte cells; (c) assembling a linear DNA expression amplicon template with an expression cassette for a desired CAR, TCR and/or antibody necessary to elicit an immune response from the subject based on the ascertained TAA, TSA, neoepitope, other epitope, neoantigen, mutational associated neoantigens, oncoviral, oncofetal, lineage-restricted, and/or over-expressed tumor antigens of therapeutic relevance; (d) amplifying the linear DNA expression amplicon template via PCR to create a plurality of linear DNA expression amplicons; (e) optionally, verifying the sequence of the plurality of linear DNA expression amplicons via NGS; (f) transfecting the population of lymphocyte cells with the plurality of linear DNA expression amplicons; (g) optionally, verifying the RNA expressed by the transfected lymphocyte cell population via NGS; and (h) optionally, verifying the structure of antibody, TCR and/or CAR expressed by the transfected lymphocyte cell population via mass spectrometry.

A specific antigen, antibody, TAA, TSA, neoepitope, other epitope, neoantigen, mutational associated neoantigens, oncoviral, oncofetal, lineage-restricted, and/or over-expressed tumor antigens of therapeutic relevance to a subject can be ascertained via any method known in the art. One or more TAA, TSA, neoepitope, other epitope mutational associated neoantigens, oncoviral, oncofetal, lineage-restricted, and/or over-expressed tumor antigens of therapeutic relevance to a subject may be identified by differential sequencing of a subject's tumor versus wild-type samples, using exome/genome sequences and RNAseq analysis, and the assistance of artificial intelligence, machine learning, predictive algorithms or the like. Through this method, a linear DNA expression amplicon comprising one or more expression cassettes encoding a CAR, TCR, antibody or other protein/polypeptide against the identified TAA, TSA, neoepitope, other epitope, neoantigen, mutational associated neoantigens, oncoviral, oncofetal, lineage-restricted, and/or over-expressed tumor antigens can be assembled and amplified via PCR or large-scale PCR. This linear DNA expression amplicon, when transfected into an appropriate lymphocyte cell and expressed by said lymphocyte cell will elicit a specific immune response based on the identified TAA, TSA, neoepitope, other epitope, neoantigen, mutational associated neoantigens, oncoviral, oncofetal, lineage-restricted, and/or over-expressed tumor antigens, resulting in a modified lymphocyte cell with limited on-target off-tumor effects and high efficacy. A single linear DNA expression amplicon may encode one CAR, TCR, antibody or other protein/polypeptide targeting a single TAA, TSA, neoepitope, other epitope, neoantigen, mutational associated neoantigens, oncoviral, oncofetal, lineage-restricted, and/or over-expressed tumor antigens or it may contained several expression cassettes for more than one CAR, TCR, antibody of other protein/polypeptide targeting more than one TAA, TSA, neoepitope, other epitope, neoantigen, mutational associated neoantigens, oncoviral, oncofetal, lineage-restricted, and/or over-expressed tumor antigens.

In another aspect, the invention provides for system for the manufacture of genetically engineered lymphocyte cells for the purpose of CAR and/or TCR based therapies, said system comprised of: (a) an input of lymphocyte cells; (b) a next generation sequencing device; (c) a PCR device; (d) optionally, a mass spectrometry device; and (e) a transfection device, wherein all aspects of the system are integrated by robotic process automation.

The lymphocyte cells can be autologous, allogeneic or a combination of the two. In one embodiment, the lymphocyte cells are T-cells or NK cells. Robotic process automation may be undertaken by one or more robotic arms and/or robotic apparatuses adapted for micro fluidic tasks. Exemplary systems include, but are not limited to, the SOLO liquid handler manufactured by Hudson Robotics (Springfield, N.J.) and the Xantus robotic pipetting platform manufactured by Tecan (Mannedorf, Switzerland). Any of the components of the system may be configured for use in conjunction with a robotic micro fluidic system. The PCR device may be a continuous flow PCR device. The PCR device may also be configured to undertake large-scale PCR. The PCR device may be a continuous flow PCR device configured for large-scale PCR. In an embodiment, the system also includes a gene synthesizer apparatus.

In some embodiments, the linear DNA expression amplicon, in addition to an expression cassette for a CAR, TCR and/or antibody, may also include an expression cassette to induce the capacity of a transfected lymphocyte cell to induce IL-12 production in a target cell or cells (e.g. a tumor) and/or the expression of cell death protein 1 (PD-1). The linear DNA expression amplicon may also include an expression cassette and/or ORF, which when expressed by an allogeneic lymphocyte cell will reduce donor incompatibility, graft versus house disease and/or host versus graft rejection in a subject. This expression cassette and/or ORF, when expressed, will down regulate MHC class I cell surface expression in an allogenic lymphocyte cell.

### System for Producing Subject-Specific Antibodies Via Engineered Lymphocyte Cells

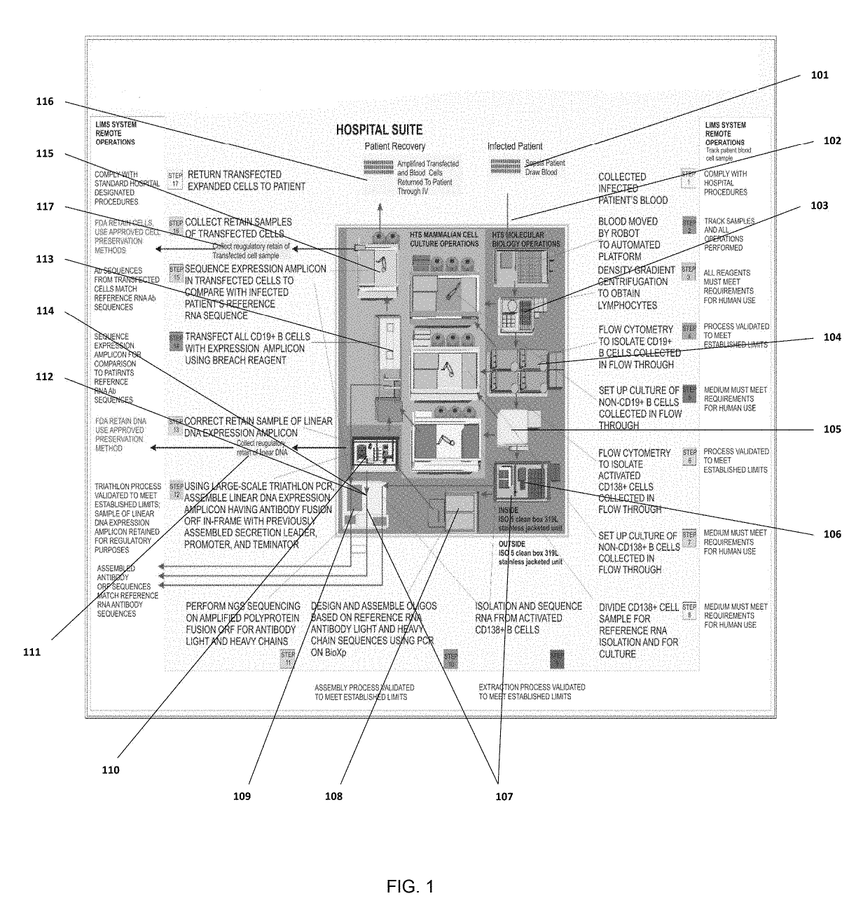

FIG. 1 is a process flow of one embodiment of the system of the invention. The system may be self-contained and configured to run without human interaction. In addition, the system may be configured for remote monitoring and control, including interconnection into various laboratory information management systems (LIMS) or other data management and storage systems for data retention, analytics and machine learning, as well as remote monitoring for QC, insurance, reimbursement and regulatory compliance. The system may be contained in an ISO 5-certified clean box or other clean room environment.

Turning to the process flow of the system disclosed in FIG. 1, a subject's blood is collected (101) and introduced into the system (102). The subject's blood, via automated means, is transferred for a density gradient centrifugation where the blood undergoes density gradient centrifugation to isolate the lymphocytes (103). The isolated lymphocytes then undergo a first round of flow cytometry to isolate CD19+B-cells (104). The remaining cells (including T helper, killer and non-CD19+B-cells) are placed in culture for expansion and storage. The isolated CD19+B-cells then undergo a second round of flow cytometry to isolate the activated CD38+ and CD138+B-cells (105). After isolation, the CD38+ and CD138+B-cells are separated into two groups, one group for RNA isolation, amplification and sequencing, and one group placed in culture for expansion and storage (106). The CD19+B-cells not containing CD38+ or CD138+ are also placed in culture for expansion and storage. The CD38+/CD138+B-cells allocated for RNA analysis are then examined for RNA expression via RNA isolation, amplification and next generation sequencing (NGS) to determine the operative RNA sequences for the light (LC) and heavy chains (HC) of the antibodies being expressed by the CD38+/CD138+B-cells in response to the subject's infection (107). Once the RNA sequences of the expressed antibodies are ascertained, one or more synthetic linear DNA open reading frame (ORF) constructs corresponding to the RNA sequences for the LC and HC of the expressed antibodies are designed and assembled via a gene synthesizer (108). The DNA sequence of the linear DNA ORF constructs are then optionally confirmed via NGS to ensure the assembled antibodies eventually “hyper” expressed by the linear DNA ORF constructs match the desired expressed antibodies of the subject (109). Once the sequence of the linear DNA ORF constructs are confirmed via NGS, the linear DNA ORF constructs undergo large scale PCR-based amplification and modification (110) to amplify the linear DNA ORF constructs and to add to each construct previously assembled expression control sequences such as a ubiquitin secretion leader, enhancer, promoter and terminator to create a quantity of linear DNA expression amplicons. A retain of the linear DNA expression amplicons may be exported from the system for preservation and regulatory compliance (111). Once amplified and assembled via large-scale PCR, the linear DNA expression amplicons are optionally sequenced via NGS (112) to confirm the error-free sequence of the linear DNA expression amplicons before transfection into the subject's cells.

The previously stored and now expanded CD38+/CD138+ and CD19+B-cells are then transfected with the linear DNA expression amplicons (113). Each transfected B-cell is isolated in its own well, expanded and a fraction is sampled for RNA expression via NGS sequencing to verify that the linear DNA expression amplicons, now transfected, encode the correct LC and HC of the desired subject specific antibodies (114). All transfected CD38+/CD138+ and CD19+B-cells with verified linear DNA expression amplicons are pooled (115). These pooled cells are then mixed with the subject's other monocyte/lymphocyte fractions that regulate antibody production, and all cells are allowed to expand in culture. This expansion allows the transfected CD38+/CD138+ and CD19+B-cells cells to stimulate helper T cells and, in addition, produces large amounts of subject-specific antibodies prior to subject reintroduction. This entire expansion cell culture set is then returned to the subject (“vein-to-vein”) (116). A retain sample of the cell culture may also be exported from the system (117). All data associated with the production run is exported to a secure data repository.

In an exemplary embodiment, a subject's blood is collected from the subject's vein into a flexible plastic United Pharma Macopharma Phlebotomy Bag 600 mL with a 16-gauge needle, (United Pharma Model: VSL7001PD) that typically contains sodium citrate, phosphate, dextrose, and sometimes adenine to keep the blood from clotting and preserve it during storage (101). The blood will be decontaminated with Plazomicin and the bags connected to the system enabling subject blood transfer (102). The blood will then be transferred inside the system into a sterile non-pyrogenic Corning® 500 mL conical polypropylene centrifuge tube with plug seal cap (CLS431123) and a maximum RCF of 6,000×g, held in Peltier thermoelectric chiller modules. The tubes will be moved by a robotic gripper arm to the indexing centrifuge (103). Lymphoprep™ (#07861), 6×500 mL, a density-gradient medium recommended for isolation of mononuclear cells from blood by exploiting differences in cell density, will be added. Granulocytes and erythrocytes have a higher density than mononuclear cells and therefore sediment through the Lymphoprep™ layer (density of 1.077 g/mL) during centrifugation. Plazomicin will be added and the tubes sealed and shaken by the robot. Each tube will be weighed so that the same amount is in each of two large centrifuge tubes for balanced centrifugation in an indexing centrifuge at 6000×g. Polyetherimide cushions provide the necessary support during centrifugation to prevent the large capacity tubes from collapsing. Centrifugation separates the blood into a top layer of clear fluid (the plasma), a bottom layer of red fluid containing most of the red blood cells, and a thin layer in between called the buffy coat (because it is buff-colored). The buffy coat layer is the fraction that contains most of the lymphocytes and monocytes. The lymphocyte population consists of T cells, B cells, and NK cells. A robotic gripper arm will return the centrifuge tubes to the chiller and unscrew the caps. A robotic pipette arm will remove the top layer and dispose of it.

The buffy coat layer will then be collected by a robotic pipette arm and placed in a BD Biosciences FACSAria clinical flow cytometer (BD Biosciences, San Jose, Calif.) which is a laser-based biological sorting device used for cell detection, counting and sorting of specific biomarkers that can excite the anti-CD19 antibody with DyLight 488 fluorophore (excitation 493 nm, fluorescence 518 nm) conjugated to it (104/105).

Two rounds of flow cytometry will be undertaken to isolate CD38+/CD138+ and CD19+B-cells (104/105). During flow cytometry screened single cells will be suspended in solution and the fluorescently labeled antibodies applied. The cells will be passed single-file through a series of lasers in the cytometer that analyze the size, shape and fluorescent characteristics of each cell. Results can then be electronically gated to filter the data to separate specific cell types that bind antibodies.

The first round of flow cytometry (104) will use anti-CD19 to isolate all CD19+B-cells from the peripheral blood. The remaining non-CD19+ cells (including T helper, Natural Killer cells) will be collected in T-75 flasks containing LGM-3™ Lymphocyte Growth Medium, a chemically defined, serum-free, xeno-free media developed to support (with the addition of appropriate cytokines) the growth and support of human lymphocytes and dendritic cells. The T-75 flasks will be moved via robot to a Cellmate automated cell culture system which has been designed for the safe, high throughput production of cell-based therapies in a cGMP environment, to be maintained at 37° C. under sterile 5% CO₂. The isolated CD19+ fraction will be held in 200 mL Corning® Dulbecco's Phosphate-Buffered Saline, 1× with calcium and magnesium.

The second round of flow cytometry (105) will isolate the subject's activated CD38+CD138+B cells from the isolated CD19+ cells, which are expressing certain antibodies in response to the subject's infection, using a BD FACSAria™ III cell sorter with a pressure driven fluidics system. Positive air pressure forces sample cells through an optically gel-coupled cuvette flow cell. Hydrodynamic focusing guides cells in a single-file stream through the cuvette, where laser light intercepts the stream at the sample interrogation point. Software design makes aseptic sort setup easy and effective. In addition, after a sample tube is run, both the inside and outside of the sample injection tubing are flushed to minimize carryover. Syndecan-1/CD138 Antibody (1A3H4) [DyLight 405] absorbs at 400 nm and emits at 420 nm (blue) and will be used as the fluorophore to isolate the subject's activated CD138+/CD38+ cells because it will not have interference from the previous fluorophore. The CD19+ cells not containing the CD138+/CD38+ will be collected in T-75 flasks in 50 mL LGM-3™ Lymphocyte Growth Medium and the flasks will be moved via a robot to the Cellmate automated cell culture system to be maintained and expanded at 37° C. under sterile 5% CO₂.

After isolation, the CD138+/CD38+ cells will be collected in Dulbecco's Phosphate-Buffered Saline, 1× with calcium and magnesium, and divided in half (106). Half of the CD138+/CD38+ cells will be held in the buffer and allowed to expand in culture. The other half of the CD138+/CD38+ cells will be placed in single wells and centrifuged. The pelleted cells will be used in the PureLink® RNA Mini Kit that provides a simple, reliable, and rapid column-based method for isolating high-quality total RNA from a wide variety of sources without the need for hazardous reagents. The single-cell RNA samples in single wells will be used for next generation sequencing (NGS) and/or for RT-PCR to ascertain the HC and LC sequence of the expressed antibodies, allowing for the selection of DNA for the ORF coding of each HC and LC (107). Whole-transcriptome analysis with total RNA sequencing (RNA-Seq) detects coding plus multiple forms of noncoding RNA. Total RNA-sequencing can accurately measure gene and transcript abundance, and identify known and novel features of the transcriptome. Sequencing will be performed via NGS on a device such as a HiSeq 11lumina sequencer using RNA-Seq process (107). These large sets of hypermutated variable ORFs (coding for hyper-variable LC and HC) will yield a number of high-affinity antibodies specifically suited to treat the infection of the subject.

The RNA sequences ascertained via NGS sequencing of the subject's CD138+/CD38+ cells (107) will provide the data necessary for the automated selection and production of photolithographically produced (or other form of synthetic DNA synthesis) oligonucleotides for rapid assembly on a gene synthesizer, (108) such as a BioXp (SGI-DNA, La Jolla, Calif.), of the antibody HC and LC gene ORFs identified in the RNA sequencing ascertained via NGS analysis (107). These linear DNA ORF constructs will be amplified and modified via large-scale PCR-based processes (110). Large-scale PCR may be undertaken by any means known in the art, including, but not limited to continuous-flow PCR as disclosed in U.S. Pat. Nos. 8,293,471; 8,986,982; and 8,163,489 or batch based methods. During large-scale PCR amplification and modification, the linear DNA ORF constructs will not only be amplified, but also modified via the addition of leader, promoter and termination arms to create linear DNA expression amplicons (201). These additional DNA constructs (leader, promoter and termination arms) may be produced in a separate PCR amplification reaction, and added in bulk to the large scale PCR amplification and modification reaction. A retain sample of the final linear DNA expression amplicons are exported from the system to enable regulatory, insurance and QC compliance. Once amplified and modified via large scale PCR, the linear DNA expression amplicons are optionally sequenced via NGS on a device such as a HiSeq Illumina sequencer (112) to confirm the sequence of linear DNA expression amplicons before transfection into the subject's cells.

The previously stored and now expanded CD38+/CD138+ and CD19+B-cells are then transfected (113) with the linear DNA expression amplicons whose sequences have been confirmed by NGS (112). Transfection of the subject's B-cells is accomplished via any known cellular transfection methodology known in the art, including, but not limited to, lipofection, electroporation, microinjection, sonication, cationic lipid delivery, cationic polymer delivery, lipid-mediated delivery, magnet-mediated delivery, gene gun or viral vector. Transfection may take place within the same large scale PCR device used to create the linear DNA expression amplicons, or it may take place in an additional large scale PCR device. In one embodiment, both amplification and modification of the linear DNA ORF constructs to create the linear DNA expression amplicons, and their subsequent transfection of the subject's CD38+/CD138+ and CD19+B-cells with said linear DNA expression amplicons take place in one or more continuous flow large scale PCR apparatuses.

The subject's transfected B-cells are then allowed to recover and expand in medium. Thereafter, the cells are transferred to individual wells via automated means. A sample of each transfected clone from each individual well is optionally used for RNA extraction, amplification and NGS sequencing (114) to verify the RNA expressed by the transfected linear DNA expression amplicons match the original RNA reference sequences (107) obtained from the subject's B-cells. This will ensure the absence of PCR-induced sequence error and will provide data that will show the linear DNA expression amplicons were correctly assembled.

Next, all transfected B-cells containing the verified linear DNA expression amplicons will be pooled (115). The other monocyte fractions containing, including but not limited to, T helper (1 and 2) cells and T natural killer (NK) cells will be combined and expanded in culture. This expansion will allow the newly transfected B-cells to stimulate other T helper cells and, in addition, will produce large amounts of subject-specific antibodies. Optimized separately produced anti-lipopolysaccharide (LPS) antibodies may also be added to the culture. This entire expansion cell culture set will be returned to the subject (116). The cell culture may be diluted or concentrated prior to reintroduction to the subject. A retain sample of the final transfected cells may be exported from the system for storage and to enable QC, insurance and regulatory compliance (117).

The sequencing and/or other data obtained from the NGS of the unmodified isolated subject's B-cells, the amplicons and the subject's transfected B-cells will be exported from the system to a remote database. The data may also be exported to a LIM system. All data will be available for review by regulatory agencies. In addition, the data may be analyzed by machine learning systems for additional data interpretation and the discovery of additional linear DNA expression amplicons and/or antibodies for use in a specific subject or specific class of subject, or as a broad therapeutic for the general population.

Inside the systems disclosed herein, automated movement and manipulation of the samples may be undertaken by one or more robotic arms and/or robotic apparatuses adapted for micro fluidic tasks. Exemplary systems include, but are not limited to, the SOLO liquid handler manufactured by Hudson Robotics (Springfield, N.J.) and the Xantus robotic pipetting platform manufactured by Tecan (Mannedorf, Switzerland). Any of the components of the system may be configured for use in conjunction with a robotic micro fluidic system.

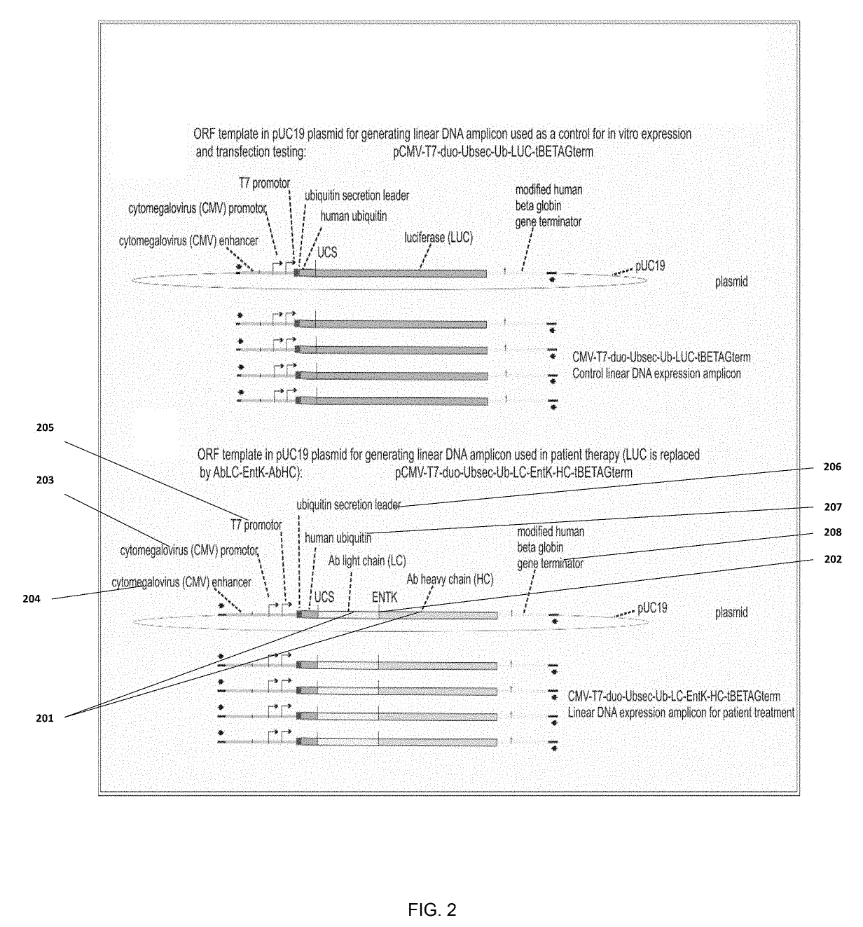

FIG. 2 shows the components of an exemplary embodiment of a linear DNA expression amplicon utilized in the system. The linear DNA expression amplicon is comprised of the ORF for the antibody LC and HC contained on the same linear DNA construct (201), split by a trypsin site for enterokinase K (ENTK) (202). In addition, the linear DNA expression amplicon is also comprised of a cytomegalovirus (CMV) promoter (203), CMV enhancer (204), T7 promoter (205), ubiqutin secretion leader (206), human ubiquitin (207) and a modified human beta globin gene terminator (208). The CMV promoter (203) and enhancer (204) will be used for immediate high-level expression of the amplicon. Use of an in-frame ubiquitin protein (206/207) increases expression of stable antibody proteins with correct folding, and may be used with an anti-infective disease-specific check point deubiquitinase inhibitor to allow very high levels of active antibody to be expressed. The antibody LC and HC ORFs (201) are both in-frame separated by a trypsin site for enterokinase K (202). Using the enhancer-driven CMV promoter and the beta globin gene terminator (208), stoichiometric amounts of antibody LC and HC will be expressed by the amplicon after trypsin cleaves the EntK site. A modified human beta globin gene terminator will be in-frame with the antibody HC ORF. As a check, the T7 promoter (205) allows expression using in vitro transcription/translation for screening of the variants.

In another embodiment, the system may be used for any form of personalized medicine and may be configured to produce modified subject cells for in vivo expression of hormones, cytokines, interleukins, interferons, erythropoietins, tumor necrosis factor and/or granulocyte/macrophage cell stimulating factor. In these embodiments, the system is configured to transfect the specific types of cells necessary for the desired expression in-vivo with linear DNA expression amplicons. Moreover, embodiments of the system may be configured to directly modify a subject's other lymphocyte cells through transfection with customized linear DNA expression amplicons.

Method for Producing Subject-Specific Antibodies Via Engineered Lymphocytes

A method for producing one or more subject-specific antibodies in vivo via linear DNA amplicons is disclosed, said method comprised of the following steps: (a) obtaining a sample of a subject's blood; (b) isolating a class or classes of lymphocyte cells from the subject's blood; (c) generating, via a gene synthesis apparatus, one or more linear DNA ORF constructs associated with the one or more desired antibodies for expression in vivo; (d) amplifying and modifying the one or more linear DNA ORF constructs to create one or more linear DNA expression amplicons; (e) optionally, verifying the sequence of the one or more linear DNA expression amplicons; (f) transfecting the subject's isolated lymphocyte cells with the one or more linear DNA expression amplicons; (g) optionally, verifying the expressed RNA sequences of the one or more transfected lymphocyte cells; (h) optionally, verifying the structure of the antibody produced by the transfected lymphocyte cells via mass spectrometry; (i) pooling the verified transfected lymphocyte cells; and (j) and reintroducing the transfected lymphocyte cells into the subject.

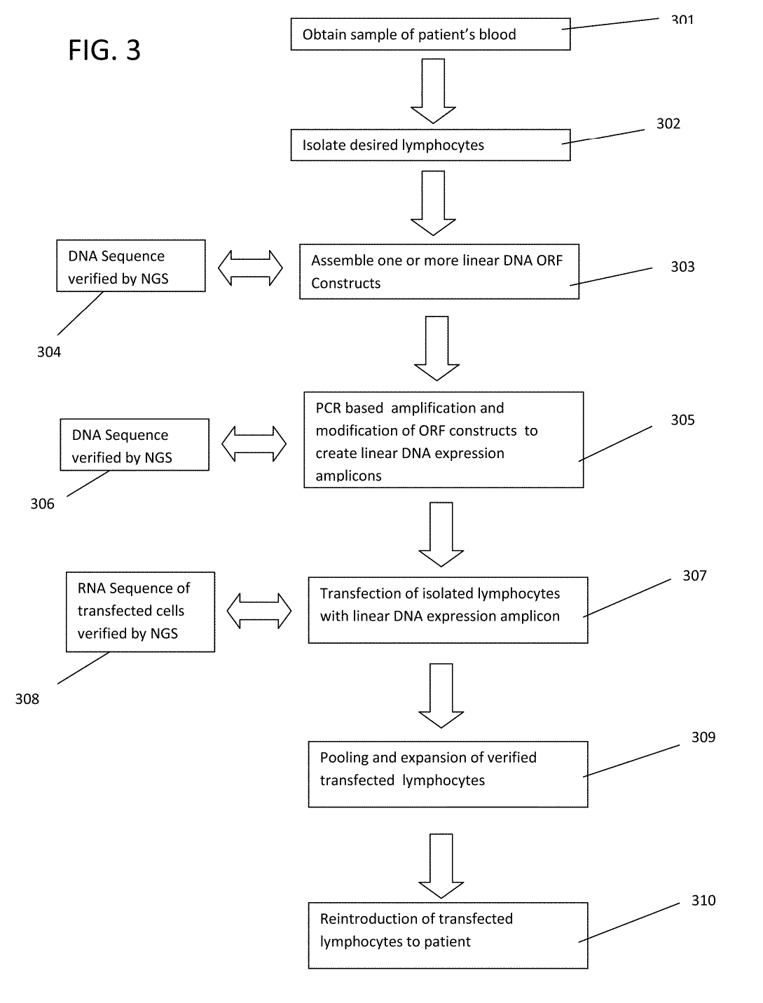

FIG. 3 is a flow diagram of an embodiment of the method of the present invention. A sample of a subject's blood is obtained (301) via standard medical methodologies. Enough blood must be obtained such that lymphocyte isolation and lymphocyte cell selection can be performed. Next, the desired lymphocytes are isolated from the subject blood (302) via centrifugation and further cell-specific isolation performed via flow cytometry to isolate a desired specific class or classes of lymphocyte cells. After, or contemporaneously with cell isolation, one or more linear DNA ORF constructs, specifically configured to express a desired antibody LC and HC in vivo, are generated via a gene synthesis apparatus (303). Gene synthesis may be performed via photolithographic means or any other means of synthetic gene synthesis know in the art capable of producing oligonucleotides of the necessary length and fidelity. Optionally, the sequence of the linear DNA ORF constructs may be verified by NGS (304). The linear DNA ORF constructs then will undergo large-scale PCR based amplification and modification to create linear DNA expression amplicons (305). The linear DNA expression amplicons include the ORFs for the antibody LC and HC, and may also include one or more of the following expression control sequences, a cytomegalovirus (CMV) promoter, a CMV enhancer, aT7 promoter, a ubiquitin secretion leader, human ubiquitin leader and a modified human beta globin gene terminator.

Optionally, the sequence of linear DNA expression amplicons is then confirmed via NGS (306). The verified linear DNA expression amplicons are then transfected into the subject's isolated lymphocytes (307). Transfection may be accomplished via any means known in the art, including but not limited to, lipofection, electroporation, microinjection, sonication, cationic lipids delivery, cationic polymer delivery, lipid-mediated delivery, magnet-mediated delivery, gene gun or viral vector. Once transfection is complete, the RNA expressed by the subject's transfected lymphocytes is optionally analyzed by NGS to confirm expression of the proper RNA to produce the desired antibodies in vivo (308). The verified transfected lymphocytes are then pooled and expanded (309) and reintroduced to the subject (310).

A method of manufacturing an immunotherapy treatment and/or biologic therapeutic is also disclosed, said method comprising the steps of: (a) assembling a linear DNA expression amplicon template including an expression cassette for a desired antibody, CAR, TCR or other therapeutically relevant protein/polypeptide via a gene synthesis apparatus or method; (b) optionally, confirming the sequence of the assembled linear DNA expression amplicon template sequence via NGS; (c) amplifying the linear DNA expression amplicon template sequence via large-scale PCR to make a plurality of linear DNA expression amplicons; (d) optionally, confirming the sequence of the plurality of linear DNA expression amplicons via NGS; and (e) transfecting cells with the linear DNA expression amplicons. The transfected cells may be human lymphocytes or any other type of cell capable of producing or creating an immunotherapy treatment or biologic therapeutic. Examples include, but are not limited to, CHO cell lines, modified CHO cell lines, stem cell lines, or murine myeloma cells lines. The transfected cells may be autologous and/or allogenic.

### Engineered Lymphocyte Cells

Engineered lymphocytes containing a linear DNA expression amplicon are provided. The engineered lymphocytes containing a linear DNA expression amplicon may express a TCR, CAR and/or antibody. Engineered lymphocytes containing a linear DNA expression amplicon may express a combination of a TCR, CAR and/or antibody. A lymphocyte cell can be engineered via a linear DNA expression amplicon to express non-personalized antibodies in vivo. Exemplary antibodies include trastuzumab, alemtuzumab and checkpoint inhibitors, such as PD-1 inhibitors, PD-L1 inhibitors and CTLA-4 inhibitors.

In an embodiment, an isolated T cell and isolated B cell, each cell containing a linear DNA expression amplicon, said T cell expressing a CAR and/or TCR, and said B cell expressing a non-personalized antibody, are disclosed. The linear DNA expression amplicon contained in the isolated T cell may or may not be the same DNA sequence as the DNA expression amplicon contained in the isolated B cell.

In another aspect, an isolated allogenic lymphocyte cell containing a linear DNA expression amplicon with an expression cassette for a CAR, TCR and/or antibody as well as a protein to reduce MHC class 1 surface expression is disclosed. The protein to reduce MHC class 1 surface expression may be viral and/or non-viral in origin.

Having now generally described the invention, the same will be more readily understood through reference to the following examples, which are provided by way of illustration, and are not intended to be limiting of the disclosed invention, unless specified.

### EXAMPLES

### Example 1—GFP Expression in Human T Cells Via Linear DNA Expression Amplicon

Linear DNA expression amplicons with an expression cassette encoding for GFP were produced from a 4707 bp GFP plasmid utilizing the materials and methods below.

| | | |
|---|---|---|
| | Final | Required |
| Description | Concentration | amount |

| | | |
|---|---|---|
| PCR water | — | 41.6 |
| Q5 5X buffer | 1X | 16 |
| 5X GC enhancer | 1X | 16 |

| | | | |
|---|---|---|---|
| dNTP 40 mM | 0.8 | mM | 1.6 |
| Q5 Polymerase 2 U/ul | 0.02 | U/uL | 0.8 |
| PS-mGFP-F-YS (100 μM) | 0.5 | uM | 0.4 |
| PS-mGFP-R-YS2 (100 μM) | 0.5 | uM | 0.4 |

| | | |
|---|---|---|
| mGFP plasmid 1 ng/μl | 1 ng/25 uL | 3.2 |
| Total volume | mL | 80 |

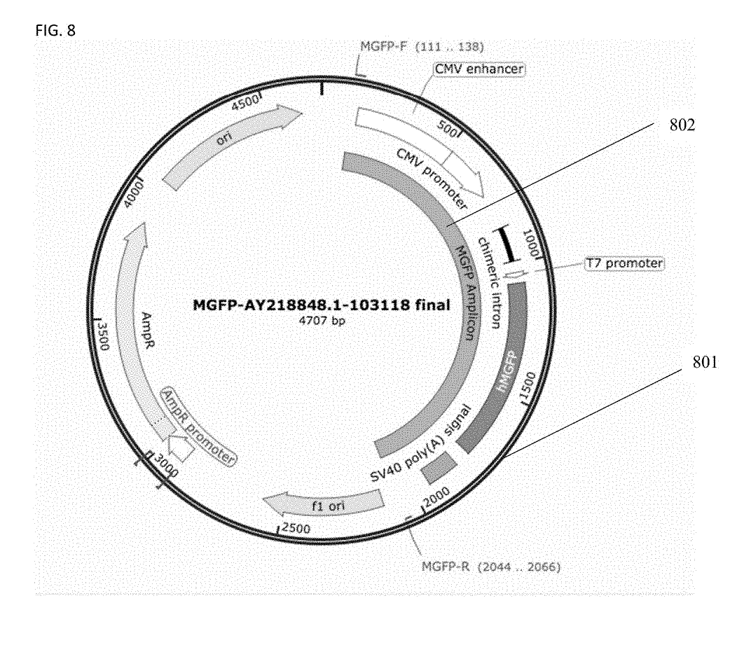

The linear DNA expression amplicon template was excised from the GFP plasmid as shown in FIG. 8. The sequence of the linear DNA expression amplicon template (802) was excised from the GFP plasmid (801), producing a linear DNA expression amplicon template of 1896 bp comprised only of the GFP ORF and necessary expression control sequences for expression of the GFP ORF in lymphocyte cells.

The PCR protocol for the high-fidelity amplification of the linear DNA expression amplicon template encoding GFP was as follows:

| PCR protocol for GFP | | | Annealing/ | Final |
|---|---|---|---|---|
| | Initial Denature | Denature | Extension | Extension |
| PCR program | 98° C. | 98° C. | 70° C. | 70° C. |
| Duration | 30 s | 15 s | 2 min | 2 min |

| | | | |
|---|---|---|---|
| Cycles | 1 | 30 | 1 |

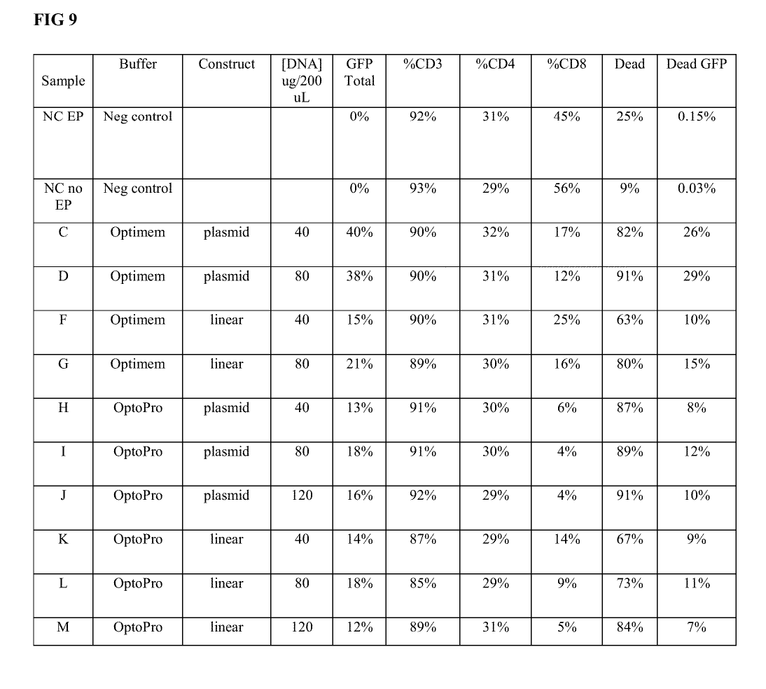

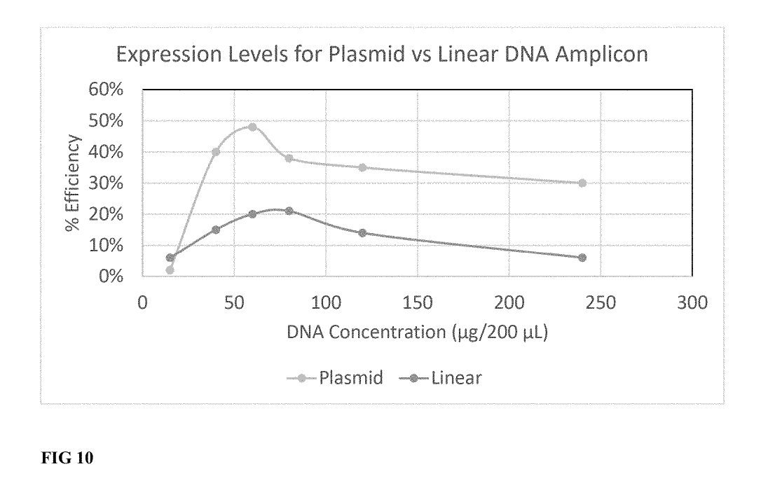

After high-fidelity PCR amplification, a large quantity of linear DNA amplicons encoding GFP were observed and confirmed via agarose gel. The linear DNA amplicons were then transfected at varying DNA concentrations into human 1×10⁷ T cell populations via a electroporation. Electroporaton was undertaken via a Maxcyte ATX® Scalable Transfection System pursuant to parameters supplied by the manufacture. Post transfection, the T cells were observed at the 48 hour period via flow cytometry and fluorescence-activated cell sorting (FACS) to ascertain GFP expression levels and cell viability. GFP expression levels at 48 hours post transfection for various linear DNA expression amplicon concentrations are summarized in FIGS. 9 and 10. Cell viability data can also be seen in FIG. 9. The highest level of GFP expression via linear DNA expression amplicons in human T cells was observed at a linear DNA expression amplicon concentration of approximately 80 ug/200 ul, though concentrations of between 40 ug/200 ul and 120 ug/200 ul all resulted in GFP expression.

### Example 2—Anti-CD19 CAR Expression in Human T Cells Via Linear DNA Expression Amplicon

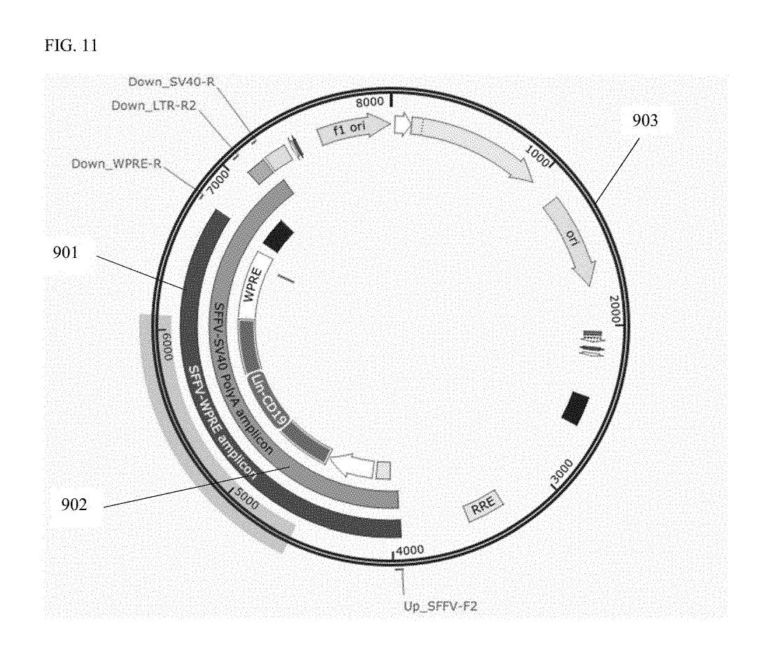

As shown in FIG. 11, two different linear DNA expression amplicons (901 and 902) containing only the anti-CD19 CAR ORF and different expression control sequences were manufactured from a 8707 bp template plasmid (903). One linear DNA expression amplicon encoding the anti-CD19 CAR was 2855 bp (901), while the second linear DNA expression amplicon with different expression control sequences but also encoding the anti-CD19 CAR was 3723 bp (902). The two linear DNA expression amplicons encoding the anti-CD19 CAR were manufactured utilizing the materials and methods below.

| | | |
|---|---|---|
| | | Required |
| Description | Concentration | amount |

| | | |
|---|---|---|
| PCR water | — | 20.8 |
| Q5 5X buffer | 1X | 8 |
| 5X GC enhancer | 1X | 8 |

| | | | |
|---|---|---|---|
| dNTP 40 mM | 0.8 | mM | 0.8 |
| Q5 Polymerase 2 U/ul | 0.02 | U/uL | 0.4 |
| PS-Up_SFFV-F2 (100 μM) | 0.5 | uM | 0.2 |
| PS-Down_WPRE-R (100 μM) | 0.5 | uM | 0.2 |

| | | |
|---|---|---|
| CD19 plasmid | 1 ng/25 uL | 1.6 |
| Total volume | mL | 40 |

The PCR protocols for the high-fidelity amplification of the two different linear DNA expression amplicons encoding anti-CD19 CAR were as follows:

| PCR protocol for CD19version 1 | | | Annealing/ | Final |
|---|---|---|---|---|
| | Initial Denature | Denature | Extension | Extension |
| PCR program | 98° C. | 98° C. | 72° C. | 72° C. |
| Duration | 30 s | 30 s | 5 min | 2 min |

| | | | |
|---|---|---|---|
| Cycles | 1 | 30 | 1 |

| PCR protocol for CD19 version 2 | | | Annealing/ | Final |
|---|---|---|---|---|
| | Initial Denature | Denature | Extension | Extension |
| PCR program | 98° C. | 98° C. | 72° C. | 72° C. |
| Duration | 30 s | 10 s | 5 min | 3 min |

| | | | |
|---|---|---|---|
| Cycles | 1 | 28 | 1 |

After high-fidelity PCR amplification of the two different linear DNA expression amplicons encoding anti-CD19 CAR, a large quantity of each linear DNA amplicon was observed and confirmed via agarose gel.

Both linear DNA amplicons were then transfected at varying DNA concentrations into separate human 1×10⁷ T cell populations via electroporation. Electroporation was undertaken via a Maxcyte ATX® Scalable Transfection System pursuant to parameters supplied by the manufacturer. Post transfection, the transfected cells were analyzed at 24 and 48 hour periods via flow cytometry and fluorescence-activated cell sorting (FACS) to ascertain anti-CD19 CAR expression and cell viability. Anti-CD19 CAR expression was observed in the transfected T cell populations.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention.

1. An engineered lymphocyte cell comprising:
a linear DNA expression amplicon, wherein said amplicon includes an expression cassette for a CAR, TCR and/or antibody.

2. The engineered lymphocyte cell of claim 1, wherein the lymphocyte cell is a T cell, B cell or NK cell.

3. The engineered lymphocyte cell of claim 1, wherein the linear DNA expression amplicon is derived from large-scale PCR.

4. The engineered lymphocyte cell of claim 2, wherein the linear DNA expression amplicon further comprises a centromere, telomere or origin or replication.

5. The engineered lymphocyte cell of claim 1, wherein the lymphocyte cell is autologous.

6. The engineered lymphocyte cell of claim 1, wherein the lymphocyte cell is allogeneic.

7. The engineered lymphocyte cell of claim 6, wherein the linear DNA expression amplicon also includes an expression cassette for a protein to reduce MHC class 1 surface expression.

8. A method for the production of engineered lymphocyte cells, wherein the method comprises:
providing a plurality of lymphocyte cells;
assembling a linear DNA expression amplicon template expressing a desired CAR, TCR and/or antibody;
amplifying the linear DNA expression amplicon template via PCR to create a plurality of linear DNA expression amplicons;
verifying the sequence of a partial quantity of linear DNA expression amplicons via NGS; and
transfecting the plurality of lymphocyte cells with the remaining quantity of linear DNA expression amplicons.

9. The method of claim 8 further comprising the steps of performing in vitro transcription on a partial quantity of the linear DNA expression amplicons to produce RNA and verifying the RNA sequence via NGS;

10. The method of claim 9 further comprising the steps of performing in vitro translation on the RNA produced via in vitro translation to produce a CAR, TCR and/or antibody protein and confirming the protein structure of the produced CAR, TCR and/or antibody via mass spectrometry.

11. The method of claim 8 wherein the plurality of lymphocyte cells are autologous.

12. The method of claim 8 wherein the plurality of lymphocyte cells are allogeneic.

13. The method of claim 8 wherein the linear DNA expression amplicon template is amplified via large-scale PCR.

14. The method of claim 8 wherein the linear DNA expression amplicon further comprises a centromere, telomere or origin or replication.

15. A method of treating cancer, said method comprising:
administering to a subject in need thereof, an engineered lymphocyte cell comprising a linear DNA expression amplicon,
wherein said amplicon includes an expression cassette for a CAR, TCR and/or antibody.

16. The method of claim 15, wherein the linear DNA expression amplicon is derived from large-scale PCR.

17. The method of claim 15, wherein the linear DNA expression amplicon further comprises a centromere, telomere or origin or replication.

18. The method of claim 15, wherein the engineered lymphocyte cell is a T cell.
